SEQUENCE LISTING (2 sequences)

SEQ ID NO: 1 - DNA, 38 bp, Homo sapiens
acgatcttaagcttccccacagtctcatcatggttcca

SEQ ID NO: 2 - DNA, 38 bp, Homo sapiens
acggtaacggatcccagtagtgggagtcggaaaacccc

(19) United States
(12) Patent Application Publication
(10) Pub. No.: US 2008/0193441 A1
(43) Pub. Date: Aug. 14, 2008

(54) Homogeneous Preparations of Chimeric Protein

(75) Inventors: Patrick W. Trown, Danville, CA (US); Michael I. Sherman, Glen Ridge, NJ (US); Kirk Dornbush, Atlanta, GA (US)

Correspondence Address: BANNER & WITCOFF, LTD., 1100 13th STREET, N.W., SUITE 1200, WASHINGTON, DC, 20005-4051, US

(73) Assignee: ICONIC THERAPEUTICS, INC., ATLANTA, GA (US)

(21) Appl. No.: 10/578,670

(22) PCT Filed: Nov. 10, 2004
(86) PCT No.: PCT/US2004/035517

§ 371 Date: Sep. 25, 2007

Related U.S. Application Data
(60) Provisional application No. 60/520,644, filed on Nov. 18, 2003.

(51) Int. Cl. A61K 39/395; C07K 16/00; A61P 9/00

(52) U.S. Cl. 424/133.1; 530/387.3

(57) ABSTRACT

Variant forms of chimeric protein molecules comprising a Factor VII moiety and an Fe region of an IgG1 moiety provide improved properties. The variants are more resistant to proteolytic degradation. Thus preparations of the variant forms are more homogeneous and have a longer half-life. The variant forms are used for treating cancer, atherosclerosis, psoriasis, diabetic retinopathy, wet macular degeneration, and rheumatoid arthritis.

### FIELD OF THE INVENTION

The invention relates to the field of immunotherapy. More particularly, it relates to the use of chimeric proteins comprising a targeting moiety and a cytolytic moiety.

### BACKGROUND OF THE INVENTION

Several prevalent diseases are associated with abnormal angiogenesis and formation of a pathological neovasculature (PNV), notably cancers with solid tumors, diabetic retinopathy, and the exudative (wet) form of age-related macular degeneration (AMD). Two procedures have been described as potential treatments for PNV-associated diseases, an antiangiogenesis protocol to inhibit angiogenesis (Folkman, J. (1995) N. Engl. J. Med. 333, 1757-1763; Kaplan, H. J., Leibole, M. A., Tezel, T. & Ferguson, T. A. (1999) Nat. Med. 5, 292-297) and an anti-PNV protocol to destroy selectively the PNV (Hu, Z. & Garen, A. (2000) Proc. Natl. Acad. Sci. USA 97, 9221-9225; Hu, Z. & Garen, A. (2001) Proc. Natl. Acad. Sci. USA 98, 12180-12185; Birchler, M. , Viti, F., Zardi, L., Spiess, B. & Neri, D. (1999) Nat. Biotechnol. 17, 984-988). Because a PNV usually has formed by the time the disease is diagnosed, destruction of the PNV probably will be necessary to achieve optimal therapeutic response.

A chimeric, antibody-like molecule, called an Icon, has been found to bind with high affinity and specificity to the receptor known as tissue factor (TF). TF is expressed on endothelial cells lining the luminal surface of a PNV but not of a normal vasculature (Drake, T. A., Morrissey, J. H. & Edgington, T. S. (1989) Am. J. Pathol. 134, 1087-1097; Contrino, J., Hair, G., Reutzer, D. L. & Rickles, F. (1996) Nat. Med. 2, 209-215), thus providing a selective and accessible therapeutic target. The Icon is composed of factor VII (fVII), the natural ligand for TF, at the N-terminus of the Icon molecule, fused to the Fe domain of an IgG1 Ig at the C-terminus of the Icon molecule. The Icon functions similarly to an anti-TF antibody, but with considerably higher affinity than can be achieved with an anti-TF antibody. The TF-Icon complex is believed to activate a potent cytolytic immune attack mediated by natural killer cells and complement (Hsu, Z., Sun, Y., and Garen, A. (1999) Proc. Natl. Acad. Sci. USA 96, 81612-8166). Cytolysis of endothelial cells of the PNV, and possibly of other cells in the wall of a leaky PNV vessel that express TF, results in selective destruction of the PNV, as demonstrated in mouse models of solid tumors (Hsu, Z., Sun, Y., and Garen, A. (1999) Proc. Natl. Acad. Sci. USA 96, 81612-8166; Hu, Z. & Garen, A. (2000) Proc. Natl. Acad. Sci. USA 97, 9221-9225; Hu, Z. & Garen, A. (2001) Proc. Natl. Acad. Sci. USA 98, 12180-12185), and in a mouse model of wet macular degeneration (Bora, P. lB., Hu, Z., Tezel, T. H., Sohn, J.-H., Cruz, J. M., Bora, N. S., Garen, A. & Kaplan, H. J. (2003) Proc. Natl. Acad. Sci. USA 100, 2679-2684).

Native Factor VII is a zymogen. Typically, in instances of blood vessel damage, Factor VII initiates the coagulation process by binding to TF; this binding promotes cleavage of Factor VII between positions 152 and 153 to generate an activated protease, Factor VIIa (fVIIa), which continues the coagulation cascade. Jurlander et al (Jurlander, B., Thim, L., Klausen, N. K., Persson, E., Kjalke, M., Rexen, P., Jergensen, T., Ostergaard, P. B., Erhardtsen, E. & Bjorn, S. E. (2001) Sem. Thrombosis Hemostasis 27, 373-383) have demonstrated that Factor VII is susceptible to this cleavage during purification under certain conditions. In addition, Factor VII and Factor VIIa are susceptible to an additional cleavage, between positions 38 and 39, that results in a much reduced affinity for TF (Sakai, T., Lund-Hansen, T., Thim, L. & Kisiel, W. (1990) J. Biol. Chem. 265, 1890-1894).

There is a need in the art for chimeric protein molecules with improved properties, including increased resistance to degradation in the body, increased shelf-life, increased binding to TF, decreased adverse side effects, and increased therapeutic effect.

### BRIEF SUMMARY OF THE INVENTION

In a first embodiment of the invention a chimeric protein is provided. The chimeric protein comprises a first and a second polypeptide. The first polypeptide is a Factor VII or Factor VIIa polypeptide and the second polypeptide is an Fc region of a human immunoglobulin IgG1. The Factor VII or Factor VIIa polypeptide contains at least one mutant residue that prevents proteolytic cleavage between residues 38 and 39 or between residues 152 and 153.

In a second embodiment of the invention a method is provided of treating a patient having a disease associated with neovascularization. An effective amount of a chimeric protein is administered to the patient. The chimeric protein comprises a first and a second polypeptide. The first polypeptide is a Factor VII or Factor VIIa polypeptide and the second polypeptide is an Fc region of a human immunoglobulin IgG1. The Factor VII or Factor VIIa polypeptide contains at least one mutant residue that prevents proteolytic cleavage between residues 38 and 39 or between residues 152 and 153. Symptoms of the disease are ameliorated by the chimeric protein.

In a third embodiment of the invention an expression vector is provided. The expression vector encodes a secreted form of a chimeric protein. The chimeric protein comprises a first and a second polypeptide. The first polypeptide is a Factor VII or Factor VIIa polypeptide and the second polypeptide is an Fc region of a human immunoglobulin IgG1. The Factor VII or Factor VIIa polypeptide contains at least one mutant residue, which prevents proteolytic cleavage between residues 38 and 39 or between residues 152 and 153.

In a fourth embodiment of the invention a method is provided for treating a patient having disease associated with neovascularization. An effective amount of an expression vector is administered to the patient. The expression vector encodes a secreted form of a chimeric protein comprising a first and a second polypeptide. The first polypeptide is a Factor VII or Factor VIIa polypeptide and the second polypeptide is an Fc region of a human immunoglobulin IgG1. The Factor VII or Factor VIIa polypeptide contains at least one mutant residue, which prevents proteolytic cleavage between residues 38 and 39 or between residues 152 and 153. Symptoms of the disease are ameliorated by the administration of the expression vector.

In a fifth embodiment of the invention a chimeric protein is provided. The chimeric protein comprises a first and a second polypeptide. The first polypeptide is a Factor VIIa polypeptide and the second polypeptide is an Fc region of a human immunoglobulin IgG1. The Factor VIIa polypeptide contains at least one mutant residue which reduces blood coagulation activity relative to wild-type Factor VIIa.

### DETAILED DESCRIPTION OF THE INVENTION

Desirable chimeras of Factor VII or Factor VIIa and the Fc region of immunoglobulin IgG1 bind with high affinity to Tissue Factor (TF), do not initiate the clotting cascade, and are resistant to degradation in the body. Mutations in Factor VII that prevent proteolytic cleavage enhance these desirable characteristics. In particular, mutations that prevent the proteolytic cleavage between amino acid residues 152 and 153 of Factor VII markedly reduce the ability of the chimeric protein to initiate the coagulation cascade while the chimeric protein retains the ability to bind with high affinity to TF. Moreover, chimeric proteins with mutations that prevent the proteolytic cleavage between amino acids 38 and 39 maintain the high affinity binding to Tissue Factor, which is lost after that cleavage occurs. Both of these types of mutations prevent proteolytic cleavages of the chimeric protein, thus maintaining a homogeneous, therapeutically active species. These mutations contribute to improved storage stability as well as to increased half-life in the body.

Any mutation of Factor VII can be used which prevents or reduces proteolytic cleavage between residues 38 and 39 or between residues 152 and 153. Such mutations include but are not limited to mutations in codons 38 and 152. In wild type Factor VII these residues are lysine and arginine, respectively. Alanine mutations can be used to substitute for these residues and abolish cleavage. Substitutions for the arginine at residue 152 with glutamate or glutamine residues have also been found to be effective. Other residues that impact either of the proteolytic cleavage sites, e.g., due to steric hindrance, can also be used. Assays for testing for the cleavage are well known in the art. A simple assay employs the use of SDS-polyacrylamide gel electrophoresis to analyze samples that have been reduced to disrupt intermolecular and intramolecular bonds. The size of the products readily indicates whether cleavage has occurred or not, and if a cleavage has occurred, whether it is between residues 38 and 39 or between residues 152 and 153 or both.

The mutations can be used singly or in combinations with each other. Moreover, they can be used in combinations with other beneficial mutations. For example the chimeric proteins may also contain mutations in the active site of the Factor VII of Factor VIIa polypeptide. Such mutations include, but are not limited to those at residues 341 and 344 of Factor VII or Factor VIIa. In addition, the Fc portion of the chimeric protein may contain beneficial mutations that improve the properties of the protein. As a non-limiting example, certain mutations at two residues of the IgG molecule, K326 and E333, increase its complement-dependent cytotoxicity activity by increasing binding to complement constituent C1q. See Idusogie et al. (2001) J. Immunol. 166, 2571-2572. Similar mutations may be used for the same purpose within the Fc portion of the chimeric protein. Such mutations can be used in combination with other mutations in the Factor VII or Factor VIIa polypeptide.

Mutations can be introduced into a coding sequence for a chimeric protein using any technique known in the art. Preferably a site-directed mutagenesis technique is used to provide a precise mutation. Alternatively, a random mutagenesis technique is used, coupled with an assay for distinguishing between proteolysis-sensitive and proteolysis-resistant molecules.

Chimeric proteins of the invention comprise a first and a second polypeptide. The first polypeptide is a Factor VII or Factor VIIa polypeptide and the second polypeptide is an Fc region of a human immunoglobulin IgG1. The polypeptides may comprise only so much of the full proteins as are necessary for functioning in the chimeric protein. Thus the first polypeptide must have the ability to bind to tissue factor with high affinity. The second polypeptide must have the ability to mediate a complement-dependent cytotoxicity response.

One way to obtain a chimeric protein comprising a Factor VII or Factor VIIa polypeptide and an Fc region of a human immunoglobulin IgG1 is described in Hu et al., (1999) Proc. Natl. Acad. Sci. USA. 96, 8161-8166. Briefly, an expression vector encoding a fVII immunoconjugate is constructed by amplifying fVII cDNA from a cDNA library using the 5′ primer ACGATCTTAAGCTTCCCCACAGTCTCATCATGGTTCCA and the 3′ primer ACGGTAACGGATCCCAGTAGTGGGAGTCGGAAAACCCC. The amplified fVII cDNA, which contains the leader and coding sequences without a stop codon, can be cloned into the HindIII and BamHI sites of the pcDNA3.1(+) vector (Invitrogen) in-frame with a cDNA encoding the human IgG1 Fc domain (Wang, B., Chen, Y., Ayalon, O., Bender, J. & Garen, A. (1999) Proc. Natl. Acad. Sci. USA 96, 1627-1632). The vector DNA can be amplified in HB101 competent cells (Life Technologies, Grand Island, N.Y.). Mutations can be introduced into fVII or IgG1 cDNA by the procedure described in the QuickChange site-directed mutagenesis manual (Stratagene). Other techniques known in the art for making fusion proteins and introducing mutations can be used as is convenient to the individual artisan.

Chimeric proteins of the invention can be administered to a patient having a disease associated with neovascularization such as cancer, macular degeneration, rheumatoid artiritis, diabetic retinopathy, psoriasis, or atherosclerosis. Administration may be local or systemic, depending upon the type of pathological condition involved in the therapy. As used herein, the term “patient” includes both humans and other mammalian species; the invention thus has both medical and veterinary applications. In veterinary compositions and treatments, chimeric proteins can be constructed using targeting and effector domains derived from the corresponding species.

Administration of chimeric proteins can be via any method known in the art such as, for example, intravenous, intramuscular, intratumoral, subcutaneous, parenteral intrasynovial, intraocular, intraplaque, or intradermal injection. The chimeric protein can also be delivered to the patient by administration of a polynucleotide molecule which encodes the chimeric protein. For example, a clinician can administer a replication-deficient adenoviral vector, adeno-associated vector, or other viral vector carrying a DNA encoding a secreted form of the chimeric protein.

For therapeutic administration, the chimeric proteins or nucleic acids are formulated singly or as combinations of proteins, dispersed or solubilized in a pharmaceutically acceptable carrier. Suitable carriers are known in the art. Preferably they are sterile and nonpyrogenic.

The amount of chimeric protein necessary to bring about the therapeutic treatment is not fixed, and is dependent on the concentration of ingredients in the composition administered. Age, weight, and physical condition of the patient are relevant considerations for setting an appropriate dosage. Preferred compositions deliver chimeric proteins in effective amounts without producing unacceptable toxicity to the patient. Pharmaceutical compositions or formulations of the invention may include other carriers, adjuvants, stabilizers, preservatives, dispersing agents, and other agents conventional in the art.

Therapeutic effects of the chimeric proteins can be further enhanced by administering to the patient any other agents known to have a therapeutic effect on the disease being treated. As an example, cancer patients frequently respond more favorably to combinations of therapies than to single agent therapy. The chimeric proteins can be administered simultaneously with the other agents or the chimeric proteins and the other agent(s) can be added sequentially.

Anti-tumor chimeric proteins can be used for treating a variety of cancers, particularly primary or metastatic solid tumors, including but not limited to melanoma, renal, prostate, breast, ovarian, brain, neuroblastoma, colorectal, head and neck, pancreatic, bladder, and lung cancer. The chimeric proteins may be employed to target the tumor vasculature, particularly vascular endothelial cells, and/or tumor cells. The tumor vasculature offers several advantages for immunotherapy, as follows. (i) Some of the vascular targets, including tissue factor, should be the same for all tumors. (ii) Chimeric proteins targeted to the vasculature do not have to infiltrate a tumor mass in order to reach their targets. (iii) Targeting the tumor vasculature should generate an amplified therapeutic response, because each blood vessel nourishes numerous tumor cells whose viability is dependent on the functional integrity of the vessel. (iv) The vasculature is unlikely to develop resistance to a chimeric protein, because that would require modification of the entire endothelium layer lining a vessel. Unlike previously described antiangiogenic methods designed to prevent new vascular growth, chimeric proteins of the invention cytolytically destroy existing neovasculature.

Chimeric proteins of the invention are also effective for treating patients with rheumatoid arthritis, wet macular degeneration, diabetic retinopathy, psoriasis, atherosclerosis, and other diseases associated with neovascularization. Administration of a chimeric protein targeted to tissue factor by a mutated human Factor VII or Factor VIIa, that is conjugated to the Fc domain of an IgG1 immunoglobulin, can generate a cytolytic immune response against the vascular endothelial cells that invade the synovium in rheumatoid arthritis and express tissue factor. Likewise, Factor VII chimeric proteins can also be effective for treating wet macular degeneration or diabetic retinopathy because of the extensive neovascularization in those pathologic conditions. Chimeric proteins of the invention can also be effective for the treatment of atherosclerosis by generating a cytolytic immune response against cells expressing tissue factor in plaques. Finally, by destroying pathological neovascularization, chimeric proteins of the invention can suppress the excess proliferation of skin cells in psoriasis.

The disclosure of co-pending application Ser. No. 10/030,203 filed Dec. 31, 2001, is expressly incorporated herein.

While the invention has been described with respect to specific examples including presently preferred modes of carrying out the invention, those skilled in the art will appreciate that there are numerous variations and permutations of the above described systems and techniques that fall within the spirit and scope of the invention as set forth in the appended claims.

We claim:

1. A chimeric protein comprising a first and a second polypeptide wherein the first polypeptide is a Factor VII or Factor VIIa polypeptide and the second polypeptide is an Fc region of a human immunoglobulin IgG1, wherein the Factor VII or Factor VIIa polypeptide contains at least one mutant residue which prevents proteolytic cleavage between residues 38 and 39 or between residues 152 and 153.

2. The chimeric protein of claim 1 wherein the mutant residue is selected from the group consisting of amino acid residue 38 and amino acid residue 152, wherein the amino acid residue at position 38 is not a lysine and the amino acid residue at position 152 is not an arginine.

3. The chimeric protein of claim 1 or 2 wherein the mutant residue is an alanine.

4. The chimeric protein of claim 2 wherein the mutant residue is a glutamine at residue 152.

5. The chimeric protein of claim 2 wherein the mutant residue is a glutamate at residue 152.

6. The chimeric protein of claim 1 wherein the Factor VII or Factor VIIa polypeptide contains an active site mutation which when present in Factor VIIa reduces blood coagulation activity relative to wild-type Factor VIIa.

7. The chimeric protein of claim 6 wherein the active site mutation is selected from the group consisting of: a non-lysine at residue 341, a non-serine residue at residue 344 and combinations thereof.

8. The chimeric protein of claim 6 wherein the active site mutation is an alanine substitution.

9. The chimeric protein of claim 1 wherein the second polypeptide comprises at least one mutation in a residue selected from the group consisting of K326 and E333 as denominated in an intact immunoglobulin, wherein the mutation increases the binding of the second polypeptide to complement constituent C1q.

10. The chimeric protein of claim 9 wherein the mutation in the second polypeptide is a tryptophan residue at K326.

11. The chimeric protein of claim 9 wherein the mutation in the second polypeptide is a serine residue at E333.

12. The chimeric protein of claim 9 wherein the second polypeptide comprises two of said mutations.

13. The chimeric protein of claim 1 which is in the form of a dimer.

14. A method of treating a patient having disease associated with neovascularization comprising:
administering to the patient an effective amount of a chimeric protein comprising a first and a second polypeptide wherein the first polypeptide is a Factor VII or Factor VIIa polypeptide and the second polypeptide is an Fc region of a human immunoglobulin IgG1, wherein the Factor VII or Factor VIIa polypeptide contains at least one mutant residue which prevents proteolytic cleavage between residues 38 and 39 or between residues 152 and 153, whereby symptoms of the disease are ameliorated.

15. The method of claim 14 wherein the disease is cancer.

16. The method of claim 14 wherein the disease is wet macular degeneration.

17. The method of claim 14 wherein the mutant residue is selected from the group consisting of amino acid residue 38 and amino acid residue 152, wherein the amino acid residue at position 38 is not a lysine and the amino acid residue at position 152 is not an arginine.

18. The method of claim 17 wherein the mutant residue is an alanine.

19. The method of claim 17 wherein the mutant residue is a glutamine at residue 152.

20. The method of claim 17 wherein the mutant residue is a glutamate at residue 152.

21. The method of claim 14 wherein the Factor VII or Factor VIIa polypeptide contains an active site mutation which when present in Factor VIIa reduces blood coagulation activity relative to wild-type Factor VIIa.

22. The method of claim 21 wherein the active site mutation is selected from the group consisting of: a non-lysine at residue 341, a non-serine residue at residue 344 and combinations thereof.

23. The method of claim 21 wherein the active site mutation is an alanine substitution.

24. The method of claim 14 wherein the second polypeptide comprises at least one mutation in a residue selected from the group consisting of K326 and E333 as denominated in an intact immunoglobulin, wherein the mutation increases the binding of the second polypeptide to complement constituent C1q.

25. The method of claim 24 wherein the mutation in the second polypeptide is a tryptophan residue at K326.

26. The method of claim 24 wherein the mutation in the second polypeptide is a serine residue at E333.

27. The method of claim 24 wherein the second polypeptide comprises two of said mutations.

28. An expression vector that encodes a secreted form of a chimeric protein comprising a first and a second polypeptide wherein the first polypeptide is a Factor VII or Factor VIIa polypeptide and the second polypeptide is an Fc region of a human immunoglobulin IgG1, wherein the Factor VII or Factor VIIa polypeptide contains at least one mutant residue that prevents proteolytic cleavage between residues 38 and 39 or between residues 152 and 153.

29. The expression vector of claim 28 which is a replication-deficient adenoviral vector or adeno-associated vector.

30. The expression vector of claim 28 wherein the mutant residue is selected from the group consisting of amino acid residue 38 and amino acid residue 152, wherein the amino acid residue at position 38 is not a lysine and the amino acid residue at position 152 is not an arginine.

31. The expression vector of claim 30 wherein the mutant residue is an alanine.

32. The expression vector of claim 30 wherein the mutant residue is a glutamine at residue 152.

33. The method of claim 30 wherein the mutant residue is a glutamate at residue 152.

34. The expression vector of claim 28 wherein the Factor VII or Factor VIIa polypeptide contains an active site mutation which when present in Factor VIIa reduces blood coagulation activity relative to wild-type Factor VIIa.

35. The method of claim 34 wherein the active site mutation is selected from the group consisting of: a non-lysine at residue 341, a non-serine residue at residue 344 and combinations thereof.

36. The expression vector of claim 34 wherein the active site mutation is an alanine substitution.

37. The expression vector of claim 28 wherein the second polypeptide comprises at least one mutation in a residue selected from the group consisting of K326 and E333 as denominated in an intact immunoglobulin, wherein the mutation increases the binding of the second polypeptide to complement constituent C1q.

38. The expression vector of claim 37 wherein the mutation in the second polypeptide is a tryptophan residue at K326.

39. The expression vector of claim 37 wherein the mutation in the second polypeptide is a serine residue at E333.

40. The expression vector of claim 37 wherein the second polypeptide comprises two of said mutations.

41. A method of treating a patient having disease associated with neovascularization comprising:
administering to the patient an effective amount of an expression vector encoding a secreted form of a chimeric protein comprising a first and a second polypeptide wherein the first polypeptide is a Factor VII or Factor VIIa polypeptide and the second polypeptide is an Fc region of a human immunoglobulin IgG1, wherein the Factor VII or Factor VIIa polypeptide contains at least one mutant residue which prevents proteolytic cleavage between residues 38 and 39 or between residues 152 and 153, whereby symptoms of the disease are ameliorated.

42. The method of claim 41 wherein the disease is cancer.

43. The method of claim 41 wherein the disease is wet macular degeneration.

44. The method of claim 41 wherein the mutant residue is selected from the group consisting of amino acid residue 38 and amino acid residue 152, wherein the amino acid residue at position 38 is not a lysine and the amino acid residue at position 152 is not an arginine.

45. The method of claim 44 wherein the mutant residue is an alanine.

46. The method of claim 44 wherein the mutant residue is a glutamine at residue 152.

47. The method of claim 44 wherein the mutant residue is a glutamate at residue 152.

48. The method of claim 41 wherein the Factor VII or Factor VIIa polypeptide contains an active site mutation which when present in Factor VIIa reduces blood coagulation activity relative to wild-type Factor VIIa.

49. The method of claim 48 wherein the active site mutation is selected from the group consisting of: a non-lysine at residue 341, a non-serine residue at residue 344 and combinations thereof.

50. The method of claim 48 wherein the active site mutation is an alanine substitution.

51. The method of claim 41 wherein the second polypeptide comprises at least one mutation in a residue selected from the group consisting of K326 and E333 as denominated in an intact immunoglobulin, wherein the mutation increases the binding of the second polypeptide to complement constituent C1q.

52. The method of claim 51 wherein the mutation in the second polypeptide is a tryptophan residue at K326.

53. The method of claim 51 wherein the mutation in the second polypeptide is a serine residue at E333.

54. The method of claim 51 wherein the second polypeptide comprises two of said mutations.

55. A chimeric protein comprising a first and a second polypeptide wherein the first polypeptide is a Factor VIIa polypeptide and the second polypeptide is an Fc region of a human immunoglobulin IgG1, wherein the Factor VIIa polypeptide contains at least one mutant residue which reduces blood coagulation activity relative to wild-type Factor VIIa.

56. The chimeric protein of claim 55 which is in the form of a dimer.
